(19) United States
(12) Patent Application Publication
(10) Pub. No.: US 2026/0290600 A1
(43) Pub. Date: Sep. 24, 2026

(54) REAL-TIME HEALTH MONITORING AND USER RANKING BASED ON SENSOR DATA

(71) Applicant: Jixuan ZHONG, Sammamish, WA (US)

(72) Inventor: Jixuan ZHONG, Sammamish, WA (US)

(21) Appl. No.: 19/084,657

(22) Filed: Mar. 19, 2025

(51) Int. Cl. G16H 50/30; G16H 10/60; G16H 40/67; G16H 80/00

(52) U.S. Cl. CPC G16H 50/30; G16H 10/60; G16H 40/67; G16H 80/00

(57) ABSTRACT

Methods, apparatuses, and non-transitory computer-readable storage media are provided for monitoring a health characteristic of a plurality of users. In one method, the method includes receiving a plurality of values for the health characteristic of the plurality of users from a plurality of user devices and ranking the plurality of values using a sort algorithm. The ranking includes: determining a greatest value of the plurality of values; determining a least value of the plurality of values; determining a slope based on the greatest value and the least value; and ranking the plurality of values based at least in part on the slope. The method further includes sending the ranking to the plurality of user devices.

### TECHNICAL FIELD

This disclosure is related to health monitoring systems, methods, and devices, and in particular, to systems and platforms for remotely tracking and evaluating the health characteristics of users through body sensor data.

### BACKGROUND

With the increasing demand for proactive healthcare management, there is a growing need for systems that enable continuous, remote monitoring of individuals' health status. Traditional methods of healthcare monitoring often require in-person visits, which can be time-consuming and may not provide real-time insights into a patient's health. As a result, there is a significant opportunity for utilizing remote monitoring platforms to track and analyze health data continuously.

Recent advancements in wearable devices and sensor technology have enabled the collection of various health-related data points, such as blood pressure, blood glucose levels, oxygen saturation (SpO2), electrocardiogram (ECG) data, and motion detection. These data points provide valuable insights into an individual's overall health and can assist in identifying potential health risks early. However, managing and analyzing the large volume of data collected from multiple users poses a significant challenge.

Current systems primarily focus on measuring individual health characteristics, but they lack the capability to compare and rank health data across multiple users in real time. This limits their ability to provide valuable context for healthcare professionals and users alike. Moreover, existing systems often fail to provide timely alerts or feedback based on the comparative ranking of health data, which could assist healthcare providers in making more informed decisions or intervene when necessary.

### SUMMARY

Examples of the present disclosure provide systems, methods, and devices for monitoring and ranking health characteristics for a plurality of users in real time.

According to a first aspect of the present disclosure, a system for monitoring a health characteristic of a plurality of users is provided. The system may include: a plurality of user devices configured to measure the health characteristic of the plurality of users, a remote server communicatively coupled to the plurality of user devices, wherein the remote server includes one or more processors, and a memory configured to store instructions executable by the one or more processors. The one or more processors, upon execution of the instructions, are configured to perform following operations including: receiving a plurality of values for the health characteristic of the plurality of users from the plurality of user devices; ranking the plurality of values using a sort algorithm; and sending the ranking to the plurality of user devices. In some examples, said ranking includes: determining a greatest value of the plurality of values; determining a least value of the plurality of values; determining a slope based on the greatest value and the least value; and ranking the plurality of values based at least in part on the slope.

In some examples, determining a slope based on the greatest value and the least value includes: determining a difference between the greatest value and the least value; determining a number of the plurality of values; determining a number of intervals between the plurality of values by using the number of the plurality of values minus one; and determining the slope by dividing the difference by the number of intervals.

In some examples, ranking the plurality of values based at least in part on the slope includes: determining respective position for respective value of the plurality of values by determining a second difference by using the respective value minus the least value and dividing the second difference by the slope; and ranking the plurality of values based at least in part on the respective position for the respective value of the plurality of values.

In some examples, the one or more processors are further configured to perform operations including: generating, based on the ranking and historical data, a plurality of messages; and sending the plurality of messages to the plurality of user devices.

In some examples, receiving the plurality of values for the health characteristic of the plurality of users from the plurality of user devices includes: obtaining sensor data from a plurality of sensors associated with the plurality of users; transmitting the sensor data to the remote server; and determining a plurality of health scores based on the sensor data.

In some examples, the one or more processors are further configured to perform operations including: detecting a health score of the plurality of health scores is lower than a threshold score; and sending an alert to a device associated with a health service provider.

In some examples, the health characteristic comprises: blood pressure level, a blood glucose level, an oxygen saturation (SpO2) level, an electrocardiogram (ECG) data, motion detection system data, accelerometer data, GPRS data, or a combination thereof.

According to a second aspect of the present disclosure, a method monitoring a health characteristic of a plurality of users is provided. The method may include: receiving a plurality of values for the health characteristic of the plurality of users from a plurality of user devices; ranking the plurality of values using a sort algorithm; and sending the ranking to the plurality of user devices. In some examples, said ranking includes: determining a greatest value of the plurality of values; determining a least value of the plurality of values; determining a slope based on the greatest value and the least value; and ranking the plurality of values based at least in part on the slope.

In some examples, determining a slope based on the greatest value and the least value includes: determining a difference between the greatest value and the least value; determining a number of the plurality of values; determining a number of intervals between the plurality of values by using the number of the plurality of values minus one; and determining the slope by dividing the difference by the number of intervals.

In some examples, ranking the plurality of values based at least in part on the slope includes: determining respective position for respective value of the plurality of values by determining a second difference by using the respective value minus the least value and dividing the second difference by the slope; and ranking the plurality of values based at least in part on the respective position for the respective value of the plurality of values.

In some examples, the method further includes: generating, based on the ranking and historical data, a plurality of messages; and sending the plurality of messages to the plurality of user devices.

In some examples, receiving the plurality of values for the health characteristic of the plurality of users from the plurality of user devices includes: obtaining sensor data from a plurality of sensors associated with the plurality of users; transmitting the sensor data to the remote server; and determining a plurality of health scores based on the sensor data.

In some examples, the method further includes: detecting a health score of the plurality of health scores is lower than a threshold score; and sending an alert to a device associated with a health service provider.

In some examples, the health characteristic comprises: blood pressure level, a blood glucose level, an oxygen saturation (SpO2) level, an electrocardiogram (ECG) data, motion detection system data, accelerometer data, GPRS data, or a combination thereof.

According to a third aspect of the present disclosure, a non-transitory computer-readable storage medium having stored thereon instructions that cause a processor to execute a method for monitoring a health characteristic of a plurality of users is provided. The method may include: receiving a plurality of values for the health characteristic of the plurality of users from a plurality of user devices; ranking the plurality of values using a sort algorithm; and sending the ranking to the plurality of user devices. In some examples, said ranking includes: determining a greatest value of the plurality of values; determining a least value of the plurality of values; determining a slope based on the greatest value and the least value; and ranking the plurality of values based at least in part on the slope.

In some examples, determining a slope based on the greatest value and the least value includes: determining a difference between the greatest value and the least value; determining a number of the plurality of values; determining a number of intervals between the plurality of values by using the number of the plurality of values minus one; and determining the slope by dividing the difference by the number of intervals.

In some examples, ranking the plurality of values based at least in part on the slope includes: determining respective position for respective value of the plurality of values by determining a second difference by using the respective value minus the least value and dividing the second difference by the slope; and ranking the plurality of values based at least in part on the respective position for the respective value of the plurality of values.

In some examples, the method further includes: generating, based on the ranking and historical data, a plurality of messages; and sending the plurality of messages to the plurality of user devices.

In some examples, the method further includes: obtaining sensor data from a plurality of sensors associated with the plurality of users; transmitting the sensor data to the remote server; and determining a plurality of health scores based on the sensor data.

In some examples, the method further includes: detecting a health score of the plurality of health scores is lower than a threshold score; and sending an alert to a device associated with a health service provider.

It is to be understood that the above general descriptions and detailed descriptions below are only exemplary and explanatory and not intended to limit the present disclosure.

### BRIEF DESCRIPTION OF THE DRAWINGS

The accompanying drawings, which are incorporated in and constitute a part of this specification, illustrate examples consistent with the present disclosure and, together with the description, serve to explain the principles of the disclosure.

FIG. 1 is a flow chart showing a method for monitoring and ranking a health characteristic of a plurality of users using a sorting algorithm in accordance with some implementations of the present disclosure.

FIG. 2 schematically illustrates a health monitoring system implemented with a sorting algorithm in accordance with some implementations of the present disclosure.

FIGS. 3A-3E illustrate experimental performance comparisons between the disclosed sorting algorithm and Tim Sort.

### DETAILED DESCRIPTION

Reference will now be made in detail to example embodiments, examples of which are illustrated in the accompanying drawings. The following description refers to the accompanying drawings in which the same numbers in different drawings represent the same or similar elements unless otherwise represented. The implementations set forth in the following description of example embodiments do not represent all implementations consistent with the disclosure. Instead, they are merely examples of apparatuses and methods consistent with aspects related to the disclosure as recited in the appended claims.

The terminology used in the present disclosure is for the purpose of describing particular embodiments only and is not intended to limit the present disclosure. As used in the present disclosure and the appended claims, the singular forms “a,” “an,” and “the” are intended to include the plural forms as well, unless the context clearly indicates otherwise. It shall also be understood that the term “and/or” used herein is intended to signify and include any or all possible combinations of one or more of the associated listed items.

It shall be understood that, although the terms “first,” “second,” “third,” etc., may be used herein to describe various information, the information should not be limited by these terms. These terms are only used to distinguish one category of information from another. For example, without departing from the scope of the present disclosure, first information may be termed as second information; and similarly, second information may also be termed as first information. As used herein, the term “if” may be understood to mean “when” or “upon” or “in response to a judgment” depending on the context.

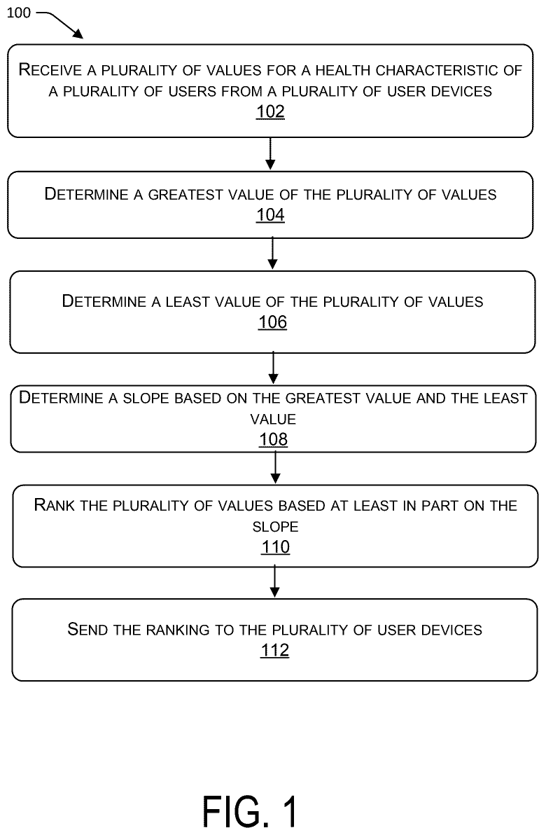

FIG. 1 illustrates a flow chart showing a method for monitoring and ranking a health characteristic of a plurality of users in accordance with some implementations of the present disclosure. In Step 102, a system may receive a plurality of values for a health characteristic of a plurality of users from a plurality of user devices. Example user devices can include, but not limited to, wearable devices such as smartwatches, fitness trackers, and health monitoring bands; mobile phones with health monitoring applications; portable health sensors; medical-grade devices like blood pressure cuffs, glucometers, and pulse oximeters; smart clothing or fabrics embedded with sensors; and other connected devices that can collect and transmit health-related data. These user devices can be equipped with a range of sensors designed to measure various health characteristics. For example, the sensors may capture a variety of health-related values, including but not limited to, blood pressure, heart rate, blood glucose levels, oxygen saturation (SpO2), electrocardiogram (ECG) data, step counts, movement data, and other physiological parameters. Each user's device continuously tracks and records these health-related values, which are then transmitted to a centralized remote server for processing.

Once the health data is received by the remote server in Step 102, the system may begin analyzing the information to determine a rank for the plurality of users. In Step 104, the system may determine a greatest value of the plurality of values. For example, the system may receive a plurality of health scores that quantifies the plurality of users' overall health based on various monitored parameters, and the system identifies the greatest health score from the received data set. For example, if the health scores of five users A-E are 10, 30, 20, 40, and 50, the system would determine that the highest health score is 50, which belongs to user E. In Step 106, the system may determine a least value of the plurality of values. In the example where the health scores are 10, 30, 20, 40, and 50, the system would determine that the lowest health score is 10, which belongs to user A.

In Step 108, the system may determine a slope based at least in part on the greatest value and the least value. The slope is calculated by determining the difference between the greatest and least health scores and dividing this by the number of intervals. This step helps normalize the health scores and creates a consistent way of ranking users relative to each other. Continue with the above example, the greatest health score is 50 and the least health score is 10. The difference between the greatest and least values is 50−10=40. Next, the number of users is 5, so there are 4 intervals. The slope is then calculated by dividing the difference by the number of intervals: slope=40/4=10. This slope value of 10 will be used to help rank the users relative to one another.

In Step 110, the system may rank the plurality of values based at least in part on the slope. For example, with health scores of 10, 30, 20, 40, and 50, the system may order the health scores for the users by determining the difference between the health score of the user from the least value 10 and dividing it by the slope 10. For user A, whose health score is 10, the system first calculates the difference from the least score (10−10=0). The system then divides this difference by the slope (0÷10=0). Based on this value, the system determines that health score 10 ranks at position 0. For user B, whose health score is 30, the system first calculates the difference from the least score (30−10=20). The system then divides this difference by the slope (20÷10=2). Based on this value, the system determines that health score 30 ranks at position 2. For user C, whose health score is 20, the system first calculates the difference from the least score (20−10=10). The system then divides this difference by the slope (10÷10=1). Based on this value, the system determines that health score 20 ranks at position 1. For user D, whose health score is 40, the system first calculates the difference from the least score (40−10=30). The system then divides this difference by the slope (30÷10=3). Based on this value, the system determines that health score 40 ranks at position 3. For user E, whose health score is 50, the system first calculates the difference from the least score (50−10=40). The system then divides this difference by the slope (40÷10=4). Based on this value, the system determines that health score 50 ranks at position 4. The system then ranks the health scores based on the positions to arrive a final ranked order of the health scores 10, 20, 30, 40, and 50. This ranking method, using the calculated slope, ensures that each user's score is normalized against the group, providing a fair comparison across all participants.

After the ranking process is completed in Step 110, the system may move on to Step 112, where the ranking is sent to the plurality of user devices. In some examples, the system may further generate messages or alerts that are sent to the user devices. These messages may inform users of their relative health standing, encourage them to improve their health status, or provide educational tips. Additionally, if any user's health score falls below a predefined threshold—indicating a potential health risk—triggers an alert to be sent to the user's healthcare provider or a designated care team. This alert serves as a notification that the user may require further evaluation or medical intervention.

In some examples, the system may also provide users with insights on their progress over time by comparing their current health score with historical data. This continuous monitoring and ranking approach enables the system to identify trends in each user's health, allowing for early intervention when necessary. Ultimately, this system helps ensure timely and proactive healthcare, providing users and healthcare providers with actionable information that can improve health outcomes.

By leveraging real-time monitoring, data analysis, and personalized feedback, the system empowers both users and healthcare providers to make more informed decisions, facilitating more effective health management and intervention strategies.

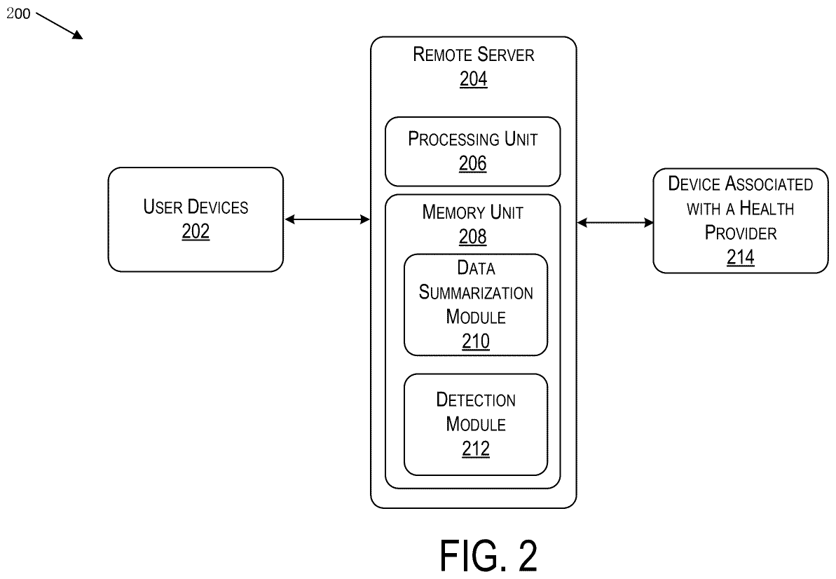

FIG. 2 schematically illustrates a health monitoring system in accordance with some implementations of the present disclosure. In various examples, a system 200 for the remote monitoring of a plurality of patients is disclosed. As shown in FIG. 2, the system 200 includes a plurality of user devices 202 configured to measure one or more health characteristics of the plurality of users, a remote server 204 communicatively coupled to the plurality of user devices 202, and a device 214 associated with a healthcare provider that is communicatively coupled to the remote server 204.

In some examples, the plurality of user devices 202, as shown in FIG. 2, includes one or more sensor units that are attached to the users' bodies to collect sensor data related to various health characteristics. These sensor units continuously or periodically monitor and record health data from the users. The health characteristics obtained by the user devices 202 may include, but are not limited to, blood pressure, blood glucose levels, oxygen saturation (SpO2), electrocardiogram (ECG) data, heart rate, body temperature, motion detection, accelerometer data, and other relevant physiological parameters. This wide range of sensor data provides a comprehensive overview of the users' health status. In some implementations, the plurality of user devices 202 are configured to analyze the collected data and generate health scores based on specific health parameters, offering a quantifiable metric of the users' health for real-time monitoring and assessment. These health scores are indicative of the users' overall well-being and can be used to track changes in health over time, enabling early detection of potential health issues. The plurality of user devices 202 may further transmit the obtained health characteristics and/or health scores to the remote server 204. In some examples, transmitting the obtained health characteristics to the remote server 204 includes transmitting over a GSM, 2G, 3G, 4G, LTE, Wi-Fi network or an Ad-hoc network created with neighboring wireless terminals. In some examples, the plurality of user devices 202 is configured to connect to other user device 214. User device 214 may be associated with health service personnel such as a specialist practitioner, emergency responder, technician, nurse, caregiver, and hospital administrator.

The remote server 204 is configured to receive, analyze, and store the data transmitted from the plurality of user devices 202. This server may be cloud-based or located within a hospital infrastructure, depending on the overall system architecture. The server includes at least one processing unit 206, which is capable of running various algorithms, such as health score sorting algorithms and predictive health analytics. These algorithms enable the system to process and interpret the health data, rank the health scores of users, and generate actionable insights regarding potential health risks.

For example, the remote server 204 may receive health scores of 63, 88, 55, and 92 from users A, B, C, and D, respectively. The server processes these scores by first identifying the greatest and least values. In this case, the greatest score is 92, belonging to user D, and the least score is 55, belonging to user C.

Next, the remote server 204 calculates a slope by finding the difference between the greatest and least health scores, which is 92−55=37. This difference is then divided by the number of intervals (3 intervals for 4 users), resulting in a slope of approximately 12.3.

With the slope calculated, the remote server 204 determines the relative positions of the users' health scores based on their relation to the least value and the slope. For user A, whose health score is 63, the server first calculates the difference from the least score (63−55=8). The remote server 204 then divides this difference by the slope (8÷12.3=0.649). Ignoring the decimal value, the remote server 204 determines that health score 63 ranks at position 0. For user B, whose health score is 88, the remote server 204 first calculates the difference from the least score (88−55=33). The remote server 204 then divides this difference by the slope (33÷12.3≈2.676). Ignoring the decimal value, the remote server 204 determines that health score 88 ranks at position 2. For user C, whose health score is 55, the remote server 204 first calculates the difference from the least score (55−55=0). The remote server 204 then divides this difference by the slope (0÷12.3=0). Based on this value, the remote server 204 determines that health score 55 ranks at position 0. For user D, whose health score is 92, the remote server 204 first calculates the difference from the least score (92−55=37). The remote server 204 then divides this difference by the slope (37÷12.3≈3.0). Ignoring the decimal value, the remote server 204 determines that health score 92 ranks at position 3.

As shown above, both user A's heath score (63) and user C's health score (55) are ranked at position 0, indicating a tie. The remote server 204 resolves this by re-ranking the tied values using a smaller slope specifically for this subset (63, 55).

For example, the remote server 204 calculate a new slope between health scores 55 and 63. Since the maximum value in this subset is 63 and the minimum value is 55, the slope is calculated as the difference between the values divided by the number of intervals, which is (63−55)/(2−1)=8. Using this new slope, the remote server 204 recalculate the rank for health scores 63 and 55. For use A, whose health score is 63, the remote server 204 first calculates the difference from the least score (63−55=8). The remote server 204 then divides this difference by the new slope (8÷8=1). This indicates health score 63 should be placed at position 1. For user C, whose health score is 55, the remote server 204 first calculates the difference from the least health score (55−55=0). The remote server 204 then divides this difference by the new slope (0÷8=0). This indicates health score 55 should be placed at position 0. Based on these positions, the remote server 204 can sort the health scores 55 and 63 in this subset.

After resolving the tie, the remote server 204 ranks the health scores for users A through D based on their final positions. To finalize the ranking, the remote server 204 first place the sorted values from the position 0 subset (health scores 55 and 63, belonging to user C and user A). The remote server 204 then add health scores 88 from position 2. Finally, the remote server 204 adds health score 92 from position 3. Thus, the remote server 204 determines the final ranked order of the health scores to be: 55, 63, 88, and 92.

Additionally, the remote server 204 is equipped with a memory unit 208 designed to securely store the rankings of the health scores, as well as other processed data. In some examples, memory unit 208 may be a non-transitory computer readable storage medium, for example, a Hard Disk Drive (HDD), a Solid-State Drive (SSD), Flash memory, a Hybrid Drive or Solid-State Hybrid Drive (SSHD), a Read-Only Memory (ROM), a Compact Disc Read-Only Memory (CD-ROM), a magnetic tape, a floppy disk and etc. This memory unit ensures that all health-related data, rankings, and analytics are stored for future reference, facilitating comparison and historical tracking. This capability allows healthcare providers or users themselves to continuously monitor health trends and take appropriate actions when needed.

In some examples, the memory unit 208 may also include a data summarization module 210. This module is configured to efficiently store and summarize the various health characteristics received from the plurality of user devices 202, ensuring that the data is organized and readily accessible for further analysis. Furthermore, the memory unit 208 may incorporate a detection module 212, which is designed to identify abnormalities in the data. For instance, if one or more health characteristics exceed a severity threshold, the detection module 212 can trigger alerts or notifications to notify healthcare providers or users of potential health concerns. This functionality helps ensure that abnormal health conditions are promptly addressed, enhancing the system's ability to facilitate proactive health management. In some examples, the severity threshold is a pre-defined severity threshold. In other examples, the severity threshold may be determined based on a patient's historical data by a machine learning model.

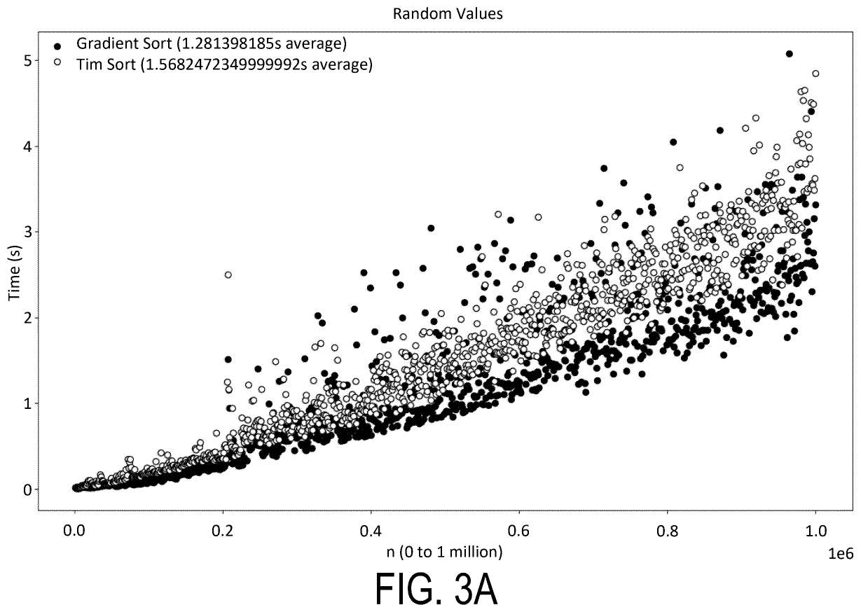

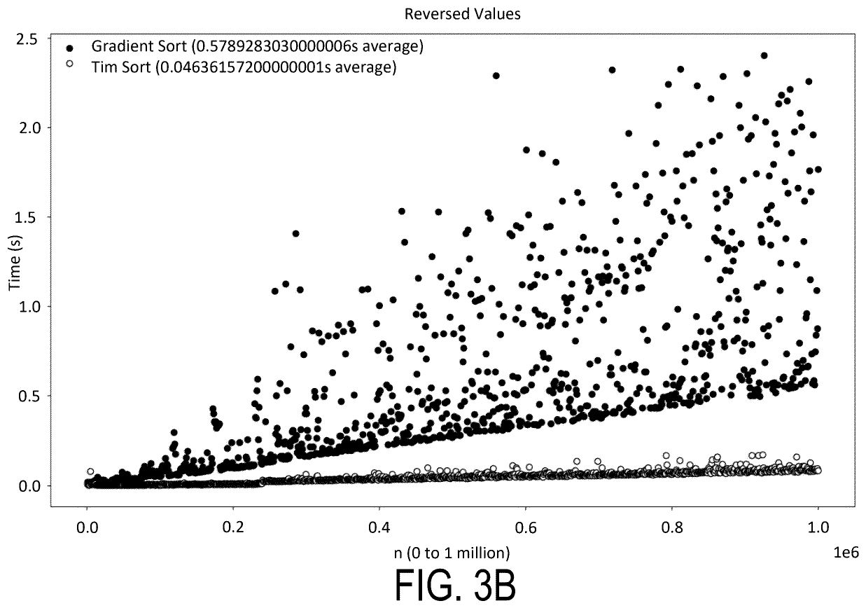

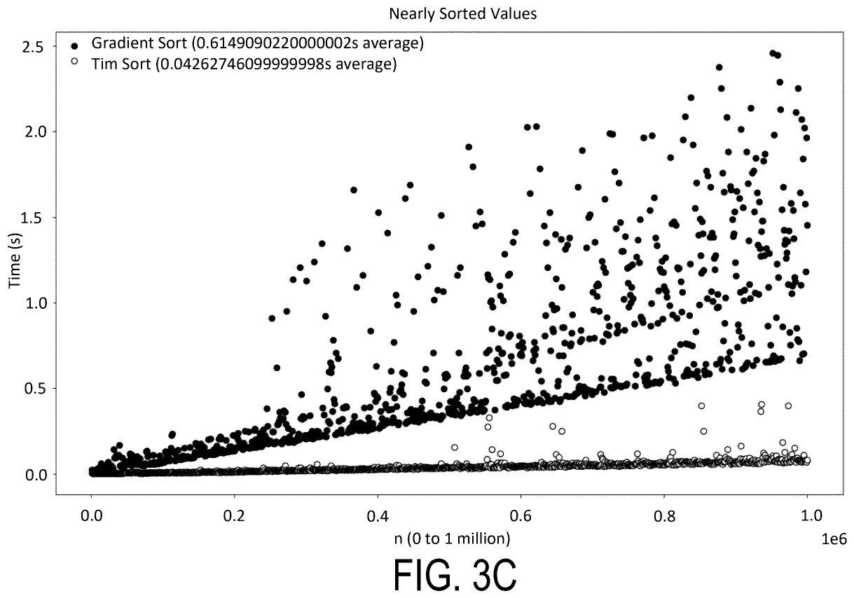

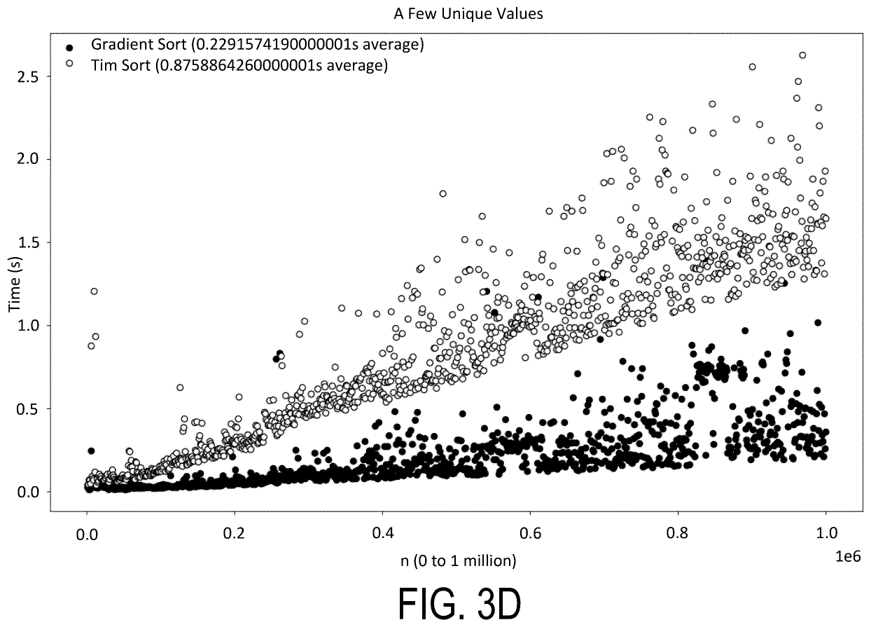

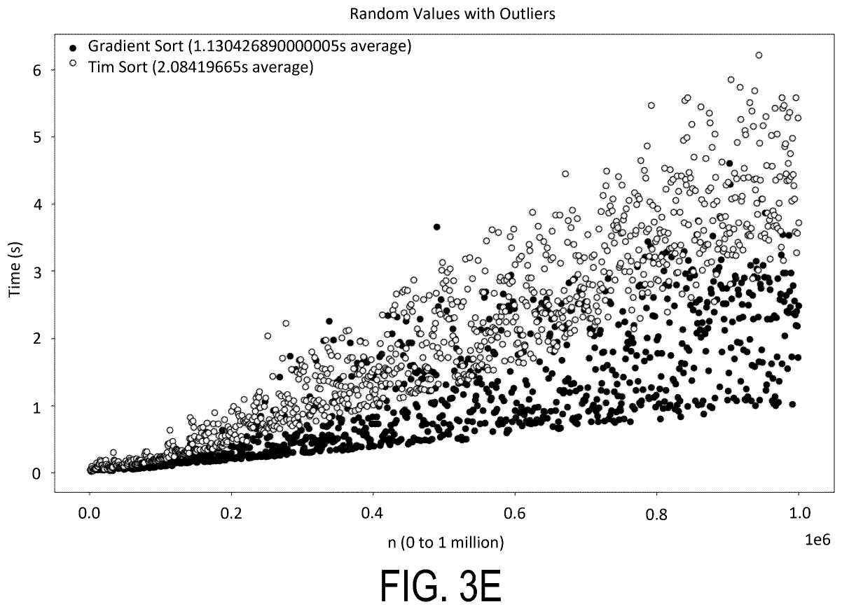

FIGS. 3A-3E illustrate experimental performance comparisons between the disclosed sorting algorithm and Tim Sort. The experiments are conducted on a dataset of one million elements, with sorting operations performed across five distinct data distributions: (1) random values, where each element is assigned a random integer within the range [0, n]; (2) reversed linear values, where elements are arranged in descending order from n to 1; (3) nearly sorted linear values, where elements are mostly sorted with occasional outliers; (4) a few unique values, where each element is randomly selected from a limited set ranging from 0 to 99; and (5) random values with outliers, where 99% of values are randomly generated within [0, n], while 1% are significantly larger. Experimental results indicate that the disclosed sorting algorithm outperforms Tim Sort in three out of five cases, demonstrating superior efficiency in handling specific data distributions.

FIG. 3A illustrates the performance characteristics of the disclosed sorting algorithm when applied to a dataset consisting of randomly generated values. In this experiment, each element within the dataset is assigned a random integer within the range [0, n], where n represents the total number of elements. As depicted in FIG. 3A, the average sorting time for the disclosed sorting algorithm is approximately 1.28 seconds, whereas the Tim Sort algorithm requires around 1.57 seconds to complete the same sorting operation. This performance disparity indicates that the disclosed sorting algorithm demonstrates superior efficiency in processing randomly distributed data. The observed improvement in sorting speed suggests that the disclosed algorithm effectively optimizes computational operations, reducing overall processing time when handling unordered datasets. FIG. 3A thus provides empirical evidence supporting the enhanced performance of the disclosed sorting algorithm over Tim Sort in scenarios involving randomly distributed numerical values

FIG. 3B illustrates the performance characteristics of the disclosed sorting algorithm when applied to a dataset consisting of reversed linear values. In this experiment, the dataset is structured such that each element is assigned a value in descending order from n to 1, where n represents the total number of elements. Specifically, each element is computed using the formula int num=n−i, ensuring a strictly decreasing sequence. As depicted in FIG. 3B, the average sorting time for the disclosed sorting algorithm is approximately 0.579 seconds, whereas the Tim Sort algorithm completes the same sorting operation in approximately 0.046 seconds.

FIG. 3C illustrates the performance characteristics of the disclosed sorting algorithm when applied to a dataset comprising nearly sorted linear values with outliers. In this experiment, the dataset consists of values predominantly arranged in ascending order from 0 to n−1, with a small proportion (approximately 1%) of outliers introduced at random positions. These outliers are significantly different from the surrounding values, disrupting the otherwise ordered sequence. As shown in FIG. 3D, the disclosed sorting algorithm requires an average of approximately 0.6149 seconds to complete the sorting process, whereas the Tim Sort algorithm executes the same operation in approximately 0.0426 seconds.

FIG. 3D illustrates the performance characteristics of the disclosed sorting algorithm when applied to a dataset containing a limited range of unique values. In this experiment, each element within the dataset is randomly assigned an integer value between 0 and 99, resulting in numerous duplicate values throughout the dataset. As shown in FIG. 3D, the disclosed sorting algorithm completes the sorting operation in approximately 0.229 seconds, whereas the Tim Sort algorithm requires an average of 0.876 seconds. The results demonstrate that the disclosed sorting algorithm significantly outperforms Tim Sort when handling datasets with a small number of unique values. This efficiency is likely due to the algorithm's ability to quickly process repeated values, reducing the number of necessary comparisons and swaps. In contrast, Tim Sort, optimized for more varied datasets, exhibits slower performance due to its reliance on merge and insertion strategies that may introduce additional computational overhead when sorting highly repetitive data. FIG. 3D highlights the superior performance of the disclosed sorting algorithm in scenarios where the dataset consists of a constrained set of unique values, making it particularly effective for applications involving categorical or low-cardinality numerical data.

FIG. 3E illustrates the performance characteristics of the disclosed sorting algorithm when applied to a dataset consisting of random values with outliers. In this experiment, approximately 99% of the dataset consists of randomly generated values within the range [0, n], while the remaining 1% comprises significantly larger values, introducing substantial irregularities in the data distribution. As shown in FIG. 3F, the disclosed sorting algorithm completes the sorting operation in approximately 1.13 seconds, whereas the Tim Sort algorithm requires an average of 2.08 seconds. The results indicate that the disclosed sorting algorithm demonstrates superior efficiency in handling datasets with extreme outliers. This improvement is likely attributed to the algorithm's ability to efficiently manage outlier positioning, reducing the impact of large values on overall sorting complexity. In contrast, Tim Sort, which relies on a combination of merge and insertion strategies, experiences increased computational overhead when adjusting for outlier values. FIG. 3E highlights the effectiveness of the disclosed sorting algorithm in processing datasets with irregular distributions, making it particularly suitable for applications where sporadic extreme values are present.

The description of the present disclosure has been presented for purposes of illustration and is not intended to be exhaustive or limited to the present disclosure. Many modifications, variations, and alternative implementations will be apparent to those of ordinary skill in the art having the benefit of the teachings presented in the foregoing descriptions and the associated drawings.

Unless specifically stated otherwise, an order of steps of the method according to the present disclosure is only intended to be illustrative, and the steps of the method according to the present disclosure are not limited to the order specifically described above, but may be changed according to practical conditions. In addition, at least one of the steps of the method according to the present disclosure may be adjusted, combined or deleted according to practical requirements.

The examples were chosen and described in order to explain the principles of the disclosure and to enable others skilled in the art to understand the disclosure for various implementations and to best utilize the underlying principles and various implementations with various modifications as are suited to the particular use contemplated. Therefore, it is to be understood that the scope of the disclosure is not to be limited to the specific examples of the implementations disclosed and that modifications and other implementations are intended to be included within the scope of the present disclosure.

1. A system for monitoring a health characteristic of a plurality of users, comprising:
a plurality of user devices configured to measure the health characteristic of the plurality of users;
a remote server communicatively coupled to the plurality of user devices, wherein the remote server includes one or more processors, and a memory configured to store instructions executable by the one or more processors, wherein the one or more processors, upon execution of the instructions, are configured to perform following operations:
receiving sensor data associated with the plurality of users from the plurality of user devices;
determining a plurality of health scores based on the sensor data;
receiving a plurality of values for the health characteristic of the plurality of users, wherein the plurality of values comprise the plurality of health scores;
ranking the plurality of values using a sort algorithm, wherein said ranking comprises:
determining a greatest value of the plurality of values;
determining a least value of the plurality of values;
determining a slope based on the greatest value and the least value; and
ranking the plurality of values based at least in part on the slope;
determining, by a machine-learning model based on historical health data associated with a first user of the plurality of users, a health-risk threshold associated with the first user;
determining that a health score associated with the first user satisfies the health-risk threshold;
in response to determining that the health score associated with the first user satisfies the health-risk threshold, generating an alert indicating a potential health condition of the first user;
transmitting the alert to a device associated with a health service provider; and
sending the ranking to the plurality of user devices.

2. The system for monitoring the health characteristic of the plurality of users of claim 1, wherein determining the slope based on the greatest value and the least value comprises:
determining a difference between the greatest value and the least value;
determining a number of the plurality of values;
determining a number of intervals between the plurality of values by using the number of the plurality of values minus one; and
determining the slope by dividing the difference by the number of intervals.

3. The system for monitoring the health characteristic of the plurality of users of claim 2, wherein ranking the plurality of values based at least in part on the slope comprises:
determining respective position for respective value of the plurality of values by determining a second difference by using the respective value minus the least value and dividing the second difference by the slope; and
ranking the plurality of values based at least in part on the respective position for the respective value of the plurality of values, wherein the plurality of values comprise the plurality of health scores associated with the plurality of users.

4. The system for monitoring the health characteristic of the plurality of users of claim 1, the one or more processors are further configured to perform operations comprising:
generating, based on the ranking and historical data, a plurality of messages indicating respective health statuses of the plurality of users; and
sending the plurality of messages to the plurality of user devices.

5. The system for monitoring the health characteristic of the plurality of users of claim 1, wherein receiving the plurality of values for the health characteristic of the plurality of users from the plurality of user devices comprises:
obtaining sensor data from a plurality of sensors associated with the plurality of users;
transmitting the sensor data to the remote server; and
determining a plurality of health scores based on the sensor data.

6. The system for monitoring the health characteristic of the plurality of users of claim 5, the one or more processors are further configured to perform operations comprising:
detecting a health score of the plurality of health scores is lower than a threshold score; and
sending an alert to a device associated with a health service provider.

7. The system for monitoring the health characteristic of the plurality of users of claim 1, wherein the health characteristic comprises: blood pressure level, a blood glucose level, an oxygen saturation (SpO2) level, an electrocardiogram (ECG) data, motion detection system data, accelerometer data, GPRS data, or a combination thereof.

8. A method for monitoring a health characteristic of a plurality of users, comprising:
receiving sensor data associated with the plurality of users from the plurality of user devices;
determining a plurality of health scores based on the sensor data;
receiving a plurality of values for the health characteristic of the plurality of users, wherein the plurality of values comprise the plurality of health scores;
ranking the plurality of values using a sort algorithm, wherein said ranking comprises:
determining a greatest value of the plurality of values;
determining a least value of the plurality of values;
determining a slope based on the greatest value and the least value; and
ranking the plurality of values based at least in part on the slope;
determining, by a machine-learning model based on historical health data associated with a first user of the plurality of users, a health-risk threshold associated with the first user;
determining that a health score associated with the first user satisfies the health-risk threshold;
in response to determining that the health score associated with the first user satisfies the health-risk threshold, generating an alert indicating a potential health condition of the first user;
transmitting the alert to a device associated with a health service provider; and
sending the ranking to the plurality of user devices.

9. The method for monitoring the health characteristic of the plurality of users of claim 8, wherein determining the slope based on the greatest value and the least value comprises:
determining a difference between the greatest value and the least value;
determining a number of the plurality of values;
determining a number of intervals between the plurality of values by using the number of the plurality of values minus one; and
determining the slope by dividing the difference by the number of intervals.

10. The method for monitoring the health characteristic of the plurality of user of claim 9, wherein ranking the plurality of values based at least in part on the slope comprises:
determining respective position for respective value of the plurality of values by determining a second difference by using the respective value minus the least value and dividing the second difference by the slope; and
ranking the plurality of values based at least in part on the respective position for the respective value of the plurality of values, wherein the plurality of values comprise the plurality of health scores associated with the plurality of users.

11. The method for monitoring the health characteristic of the plurality of users of claim 8, further comprises:
generating, based on the ranking and historical data, a plurality of messages indicating respective health statuses of the plurality of users; and
sending the plurality of messages to the plurality of user devices.

12. The method for monitoring the health characteristic of the plurality of users of claim 8, wherein receiving the plurality of values for the health characteristic of the plurality of users from the plurality of user devices comprises:
obtaining sensor data from a plurality of sensors associated with the plurality of users;
transmitting the sensor data to the remote server; and
determining a plurality of health scores based on the sensor data.

13. The method for monitoring the health characteristic of the plurality of users of claim 12, further comprising:
detecting a health score of the plurality of health scores is lower than a threshold score; and
sending an alert to a device associated with a health service provider.

14. The method for monitoring the health characteristic of the plurality of users of claim 8, wherein the health characteristic comprises: blood pressure level, a blood glucose level, an oxygen saturation (SpO2) level, an electrocardiogram (ECG) data, motion detection system data, accelerometer data, GPRS data, or a combination thereof.

15. A non-transitory computer-readable storage medium having stored thereon instructions that cause a processor to execute a method for monitoring a health characteristic of a plurality of users, the method comprising:
receiving sensor data associated with the plurality of users from the plurality of user devices;
determining a plurality of health scores based on the sensor data;
receiving a plurality of values for the health characteristic of the plurality of users, wherein the plurality of values comprise the plurality of health scores;
ranking the plurality of values using a sort algorithm, wherein said ranking comprises:
determining a greatest value of the plurality of values;
determining a least value of the plurality of values;
determining a slope based on the greatest value and the least value; and
ranking the plurality of values based at least in part on the slope;
determining, by a machine-learning model based on historical health data associated with a first user of the plurality of users, a health-risk threshold associated with the first user;
determining that a health score associated with the first user satisfies the health-risk threshold;
in response to determining that the health score associated with the first user satisfies the health-risk threshold, generating an alert indicating a potential health condition of the first user;
transmitting the alert to a device associated with a health service provider; and
sending the ranking to the plurality of user devices.

16. The non-transitory computer-readable storage medium of claim 15, wherein determining the slope based on the greatest value and the least value comprises:
determining a difference between the greatest value and the least value;
determining a number of the plurality of values;
determining a number of intervals between the plurality of values by using the number of the plurality of values minus one; and
determining the slope by dividing the difference by the number of intervals.

17. The non-transitory computer-readable storage medium of claim 16, wherein ranking the plurality of values based at least in part on the slope comprises:
determining respective position for respective value of the plurality of values by determining a second difference by using the respective value minus the least value and dividing the second difference by the slope; and
ranking the plurality of values based at least in part on the respective position for the respective value of the plurality of values, wherein the plurality of values comprise the plurality of health scores associated with the plurality of users.

18. The non-transitory computer-readable storage medium of claim 15, the method further comprises:
generating, based on the ranking and historical data, a plurality of messages indicating respective health statuses of the plurality of users; and
sending the plurality of messages to the plurality of user devices.

19. The non-transitory computer-readable storage medium of claim 15, the method further comprises:
obtaining sensor data from a plurality of sensors associated with the plurality of users;
transmitting the sensor data to the remote server; and
determining a plurality of health scores based on the sensor data.

20. The non-transitory computer-readable storage medium of claim 19, the method further comprising:
detecting a health score of the plurality of health scores is lower than a threshold score; and
sending an alert to a device associated with a health service provider.
